(19) United States
(12) Patent Application Publication
(10) Pub. No.: US 2023/0181768 A1
(43) Pub. Date: Jun. 15, 2023

(54) TISSUE-TARGETING COMPLEX

(71) Applicant: PHARMPUR GMBH, Königsbrunn (DE)

(72) Inventors: Melanie Lievenbrück, Königsbrunn (DE); Dirk-Henning Menz, Königsbrunn (DE); Helge Menz, Königsbrunn (DE); Bernd-Kristof Müller, Königsbrunn (DE)

(21) Appl. No.: 16/487,233

(22) PCT Filed: Feb. 21, 2018
(86) PCT No.: PCT/EP2018/054320

§ 371 Date: Sep. 18, 2019

(30) Foreign Application Priority Data
Feb. 21, 2017 (EP) 17157280.3

(51) Int. Cl. A61K 49/00

(52) U.S. Cl. CPC A61K 49/0021; A61K 49/0054

(57) ABSTRACT

A tissue-targeting complex is described that comprises at least one targeting element [A-L-Q⁺-Alk B-] in which A is an amino-containing anchor element, L a linker, Q⁺ a quaternary ammonium group, B⁻ an ophthalmologically acceptable counterion, and Alk an alkyl chain having 4 to 12 carbon atoms; at least one chromophore, and optionally a carrier molecule.

The present invention relates to a tissue-targeting complex and the use thereof, particularly in ophthalmology.

In the field of analytics and diagnostics, dyes for staining tissues, e.g. membranes, are an important tool. A dye suitable for staining should bind as selectively as possible to a particular type of tissue or membrane, so that the binding of this dye is a marker of its presence or location. A dye suitable for this purpose must be generally tolerated and should have a color that stands out well from the surrounding area in which staining is to be carried out.

In ophthalmology, dyes are used to stain membranes that subsequently are to be surgically removed. A dye used for this purpose should be water-soluble, as solvents should be avoided in an organ as sensitive as the eye, should have a color that allows easy differentiation from the surrounding area, should be easily removable after use, and of course should not irritate or damage the eye. Although there are already some dyes for staining membranes in the eye, all such dyes to date have drawbacks that the present invention aims to overcome.

For eye disorders such as macular degeneration, retinopathy, retinal changes, etc., vitrectomy is often indicated, i.e. a procedure in which the vitreous body or parts thereof are removed in order then to permit the removal from the retina of the inner limiting membrane or any epiretinal membranes that may have formed inside the eye, in order to induce healing processes and/or prevent further damage to the retina. In such surgery, the membrane concerned is peeled away from the retina using standard surgical instruments and it is very important for the surgeon to be able to distinguish between the membrane to be removed and the retina beneath, so as not to damage the retina during the procedure.

The retina consists of several layers with individual functions. The choroid contains the majority of the photoreceptors and is additionally responsible for adequate blood supply. It constitutes the outer limit of the retina; on the inside the retina adjoins the vitreous body. The inner limiting membrane is the basal membrane of the Muller cells, i.e. an extracellular membrane that separates the vitreous body from the retina and thus constitutes the inner limit of the retina. It is attached not only to the Muller cell processes, but also, via fibrils, to the vitreous body. This attachment is characterized by adhesions of collagen fibers in the vitreous body to the ILM. The ILM consists of different types of glycoprotein and collagen. The thickness of this layer is up to 2.5 µm. This boundary layer is often affected by disorders that cause visual impairment and may/must be treated through surgical procedures.

For example, so-called epiretinal membranes can form on the retina; these can develop in the absence of any trigger or form as a result of earlier disorders or procedures. These membranes can contract, resulting in retinal deformation and consequent visual impairment. Such deformations that pull on the retina or the macula therefore need to be removed.

ILM removal is also an option for the treatment of retinal detachment and macular degeneration. To be able to remove this fine membrane through “ILM peeling”, it is helpful to identify it with a dye that can selectively stain the tissue to be removed.

Examples of dyes known for this purpose are Brilliant Blue G, Brilliant Blue R, Patent Blue V, and Trypan blue and also Indocyanine green. These dyes show either toxic effects or insufficient staining power for the surgical procedure.

There have been past efforts to find dyes for staining membranes, which were to adhere as selectively as possible to the membrane to be stained. It was consequently very difficult to find suitable dyes for the specific staining of individual membranes that show an adequate level of selectivity allied with physiological acceptability, water solubility, and suitable shade.

It was therefore an object of the present invention to provide a staining agent that stains a membrane sufficiently for it be visible. It was also an object of the invention to provide an agent having a shade adjustable through simple substitutions. A further object of the invention was to provide a staining agent that is unable to penetrate into deeper cell layers, but is able to selectively interact in the target area such that the dye persists in the intended location in sufficient amount and for sufficiently a long period after staining.

All these objects are achieved by the tissue-targeting complex according to the invention as defined in the claims.

The invention relates to a tissue-targeting complex that comprises at least one targeting element [A-L-Q⁺-Alk B⁻] in which A is an anchor group, L a linker, Q⁺ a quaternary ammonium group, B⁻ an ophthalmologically acceptable counterion, and Alk an alkyl chain having a chain length of 4 to 12 carbon atoms; at least one chromophore, and optionally a carrier molecule.

It has now been found that, surprisingly, complexes suitable or adapted for binding to ocular tissue or for staining ocular tissue, respectively, can be formed by using a complex comprising at least two elements, namely at least one targeting element and at least one chromophore and/or at least one carrier molecule. For example, the tissue-targeting complex according to the invention may include a chromophore and additionally a carrier molecule as a third component. With this combination according to the invention, it is possible to target membranes selectively and to stain them with a dye that imparts a desired color.

The advantageous properties are achieved by providing separate units for the function of interaction with a desired environment and for the staining. This makes it possible to provide a chromophore that has a desired shade, but is not selective for a tissue to be stained, in a form that allows the corresponding tissue to be stained selectively.

### DEFINITIONS

The term “complex”, as used here, refers to an assembly of molecular components that perform different functions, including independently of one another.

A tissue-targeting complex of the present invention is a complex comprising a targeting unit and a staining or functional unit which, on contact with a target tissue, interacts there and stains the target area to make it visible to the eye, either directly or on irradiation with light of a suitable wavelength.

The term ”chromophore “ describes a molecule, an element, a unit or a group that can give rise to color either directly or through irradiation with light of a suitable wavelength. The color produced is one that is visible to the eye and is thus in the range from 380 to 780 nm. The terms “physiologically acceptable” and “ophthalmologically acceptable” refer to compounds, substances, materials, etc. that do not damage the organism to which a composition containing said compound, material or substance is administered and which are tailored to the conditions prevailing therein. The term “ophthalmologically acceptable” means, over and above this, that the substance under consideration does not irritate or damage the eye, which is particularly sensitive.

The term “carrier molecule” describes any type of molecule that is able to act as a carrier for elements such as targeting elements or chromophores according to the present invention. “Carrying”, where it refers in this context to an element, means any type of fixation to a carrier molecule, which may be through covalent bonding or through interactions, for example by adsorption. Elements are usually covalently bonded to a carrier molecule. The carrier molecule may be of any form suitable for its function. Thus, the carrier molecule may take the form of chains, balls, nano- or microparticles or other common shapes for inert carrier molecules.

The term “linker unit”, as used here, refers to a molecule that connects two groups, with the linker unit being essentially inert and neutral, meaning that the function of the elements that it connects is not affected significantly. A linker unit may be any molecule that connects two units. A linker molecule is of a length that is suitable for the particular purpose; in order for the elements to be connected not to be too far apart, the unit should not be too long. The linker unit does not normally contribute either charge or functionality.

A “quaternary ammonium ion” is a group that is positively charged and consists of a nitrogen and four groups attached to it.

The terms “water-soluble” and “water-compatible” are used interchangeably in connection with polymers and with the tissue-targeting complex according to the invention. Since the gradations between dispersion and solution often cannot be pinpointed for large molecules that contain polymers, such as the tissue-targeting complexes according to the present invention, a water-soluble/water-compatible tissue-targeting complex is understood to mean one that can be mixed with water and does not settle out.

The term “target area” refers to the area in which binding or staining is to be accomplished. For the present invention, this is normally the ILM and/or epiretinal membranes.

The term “interact”, when used in connection with the selectivity of the targeting element, describes any type of interaction between targeting element and membrane that results in the targeting element, and thus the complex according to the invention, remaining in the place where the interaction occurs. The term covers any type of physical interaction, provided the complex is held at the place at which it is to exert its effect.

The term “ocular membranes” is used to describe the membranes in the eye, particularly epiretinal membranes and the ILM.

The complex according to the invention comprises at least one targeting element that directs the complex to the target area that is to be stained and in which the dye and/or the carrier molecule is to undergo adsorption, binding or other such interaction for the desired period of time. A targeting element is therefore an element that interacts specifically with a target area.

A structural element has now surprisingly been found that interacts specifically with ocular membranes. This structural element [A-L-Q⁺-Alk B⁻] consists of an anchor group, a linker, a quaternary ammonium group, and an alkyl chain having a length of 4 to 12 carbon atoms. The anchor group is formed from a heteroatom, which may be N, O, S, P or another heteroatom suitable for attachment, or a functional group, and has the function of attachment to a chromophore or to a carrier molecule. The heteroatom may in particular be N, S or O. As the functional group, consideration is given to groups that can easily form bonds, such as alcohol radicals, carbonyl radicals, carboxyl radicals, etc.

The anchor group is connected via a linker to a quaternary ammonium group. The linker molecule is a molecule that connects the anchor group to the quaternary ammonium group, keeps the two groups a predetermined distance apart, and also contributes flexibility. Any molecule that has a chain length in the range from 1 to 10 atoms, preferably 1 to 8 and especially 1 to 6 atoms, and fulfills the function described is suitable here. A particularly suitable alkyl chain has been found to be one having 1 to 6, preferably 2 to 4 carbon atoms.

The quaternary ammonium group Q⁺, which is attached via the linker to the anchor group at one end and also bears an alkyl chain at the other end, has two further substituents. These two substituents are noncritical, provided they are neutral as regards the function of guiding the molecule to a target area, i.e. do not affect the desired function. Suitable examples are C₁-C₄ alkyl groups. The two substituents are preferably independently H or methyl.

In addition, there is a counterion B- for the quaternary ammonium group. The counterion is noncritical per se. Any physiologically or ophthalmologically, respectively, suitable counterion can therefore be used. Preference is given to the use as counterion of a halide ion, such as chloride ion or bromide ion, or hydrogen phosphate ion or hydrogen sulfate ion.

The third part of the targeting element according to the invention is an alkyl chain having a chain length of 4 to 12 carbon atoms, preferably 6 to 10 carbon atoms. It was found that an alkyl chain that is attached to the quaternary ammonium group contributes to the interaction with the ILM. If the alkyl chain has fewer than 4 carbon atoms, selective binding is no longer achieved. If the chain length exceeds 12 carbon atoms, there are problems with the water solubility of the complex according to the invention. Moreover, it has been found to be disadvantageous if the alkyl chain contains unsaturated bonds, as these restrict the flexibility of the chain. Branched alkyl chains are also suitable, provided the branching does not impair flexibility. If a branched alkyl group is used, a critical factor is the length of the longest straight chain, which is described here as the chain length. The branches present in the chain are generally methyl groups.

The targeting element may be attached via the anchor group either to a chromophore or to a carrier molecule, or to both, and bears, as a specifically binding element that is in turn attached via a linker, a quaternary ammonium ion having a flexible alkyl chain, which is essential for the interaction with ocular membranes.

Surprisingly, it was found to be precisely the combination of quaternary ammonium ion and flexible alkyl chain that results in specific interaction of the complex with ocular membranes. Without being tied to a theory, it is thought that the positive charge of the quaternary ammonium ion together with the hydrophobic alkyl chain has the result that the molecule is drawn to the ocular membranes and is held in place there as a consequence of the charge distribution and hydrophobicity. The targeting element should therefore be bound to a chromophore and/or a carrier molecule in such a way that the quaternary group and hydrophobic component are amenable to interaction with the target area. Other advantages of this interaction are that the staining agent or carrier molecule go where staining or binding is desired—- at the ocular membranes - and stays there, which means that the amount of staining agent can be reduced compared with nonspecifically adhering dyes, that there is less clouding of vision outside the target area due to nonspecifically attendant staining agent, and that a large part of the staining agent is removed when the membrane is peeled off together with the ocular membrane.

A second essential component of the tissue-targeting complex according to the invention is at least one chromophore or at least one carrier molecule, respectively. Since the staining complex is intended for staining an ophthalmic membrane, the dye or the chromophore, respectively, must meet the abovementioned requirements, i.e. it must not be irritating or toxic and it must achieve adequate staining and form a water-soluble or water-compatible staining complex.

The chromophore used may be any known dye per se, provided it meets the abovementioned requirements. Dyes in the blue to green shade range are especially suitable for staining membranes in the eye, so as to provide maximum contrast against the reddish background. In addition, the staining power of the dye must be as high as possible, as the stronger this is, the lower the concentration of the dye can be. It is desirable for the concentration to be as low as possible in order to need to use a very low dose, which also achieves better tolerability.

The complex according to the invention also helps to keep the dose lower than is the case for other known dyes. The selective staining of the desired membrane and the attachment brought about by the targeting element means that the amount of dye can be reduced. In particular, the dye is selectively brought to the desired location and remains there.

The anthraquinones group has been found to be highly suitable for the use according to the invention and as part of the complex according to the invention. Anthraquinone dyes are well known per se. The color can be adjusted through derivatization, which means that a desired shade can be achieved through appropriate derivatization. In addition, further elements such as targeting elements, bridging elements or carrier molecules can be introduced/attached.

Dyes with absorptions throughout the visible range may be synthesized by varying the nature of the substituents and the position thereof in the anthraquinone base skeleton. Particularly substitutions in the 1, 4, 5, and 8 positions, i.e. in the α-position of the anthraquinone, have a strong influence on the absorption of the dye. Unsubstituted anthraquinone has an absorption maximum at 405 nm. Electron donors cause a bathochromic shift. i.e. a shift toward longer wavelength. Thus, for example, 1-aminoanthraquinone has an absorption maximum of λₘₐₓ = 475 nm; the absorption maximum of 1,4-diaminoanthraquinone is shifted by 115 nm toward longer wavelength. This is accompanied by an increase in the extinction coefficient, which is for example three times higher in the case of 1,4-diaminoanthraquinone. Substituents in the 2, 3, 6, and 7 positions, i.e. in the so-called β-position, have a less pronounced effect on the color produced and on reactivity. These positions may optionally be used for attaching other elements. Since the shade is already altered by the introduction of a functional group, it is also possible to use one of the other α-positions for attaching further elements without restricting the color options.

Further advantages of anthraquinone dyes are their high stability to thermal effects, chemicals, and light.

The α-positions of the anthraquinone can thus be selectively derivatized to tailor it to the desired shade. Furthermore, the anthraquinone skeleton offers the option of introducing a targeting element, since there are sufficient positions available for derivatization. Derivatization can itself be accomplished relatively easily by standard methods, as described in the examples.

As described above, it is advantageous to provide dyes with a color in the blue to green range. It has been found that anthraquinones substituted with amines at the two αpositions of one of the two phenyl rings give a blue color, whereas anthraquinones in which an amino group and a halogen group are attached at the α-positions of a phenyl ring instead have a color in the reddish region. If a different heteroatom is attached, for example O and S, the color is likewise altered. In such cases, the second phenyl ring may be unsubstituted. One embodiment of the present invention therefore uses anthraquinones that are substituted with substituted amino groups at two α-positions as a chromophore to impart a blue color.

The chromophore used is in particular a compound of formula I

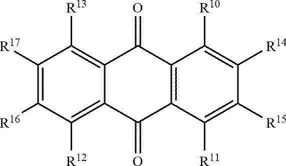

wherein each of the radicals R¹⁰, R¹¹, R¹², and R¹³ is independently hydrogen, C₁-C₄ alkyl radicals, an anchor group as defined in claim 1 or a linker molecule, each of the radicals R¹⁴, R¹⁵, R¹⁶, and R¹⁷ is independently hydrogen, a C₁-C₄ alkyl radical or a linker unit.

In one embodiment, anthraquinones are provided that not only have the preferred blue shade, but also bear targeting elements that have the property of binding selectively to ocular membranes, the ILM, and ERMs, by using the two amino groups introduced into the anthraquinone to achieve the blue color to act simultaneously as anchor groups for the targeting element. The targeting elements do not adversely affect staining and the anthraquinone group does not adversely affect specific binding. Therefore, in a preferred embodiment, the anthraquinone bears an amino group substituted with a C₁-C₆ alkyl group at two α-positions, while the other end of the alkyl group that is not attached to the anthraquinone amino nitrogen bears a quaternary ammonium ion that is in turn attached to an alkyl chain. This combination of anthraquinone dye and targeting element both imparts the blue color and contributes to the interaction at ocular membranes. A tissue-targeting complex that has many advantageous properties is thus provided.

The anthraquinone may be substituted in one to four α-positions with heteroatom groups, in particular amino groups. As described above, the number of substituents may be used here as a means of influencing the shade appropriately. If two substituents are used, they may either both be attached at the α-positions of one phenyl group or one amino group may be attached to each of the two phenyl groups.

In one preferred embodiment, the amino group is substituted such that it is simultaneously also the targeting element of the complex.

In another embodiment, an anthraquinone endowed with the desired shade through appropriate substitution is derivatized further by introducing a targeting element at one of the positions that are still free. Therefore, in another embodiment, 1, 2, 3 or 4 of the αpositions of the anthraquinone bear substituents that influence the shade, e.g. electron donors such as alkylamino groups, and one of the remaining positions, which may for example also be a β-position, bears the targeting element, attached via the anchor group.

These anthraquinones that are preferred in accordance with the invention are water-soluble or water-compatible and can therefore be used in this form for staining ocular membranes.

It has now been found that, depending on the substituents, anthraquinones are able to penetrate into the deeper regions of cells, for example in the vicinity of ocular membranes. It is generally undesirable for compounds used for staining or treatment to be able to penetrate more deeply into the cell, for example into the cell nucleus, and are able to exert cytotoxic effects. Furthermore, staining of cells that are not removed and therefore remain in the body is undesirable.

In one embodiment aimed at preventing a tissue-targeting complex according to the invention from being able to penetrate into the cell, the tissue-targeting complex contains a carrier molecule in addition to the at least one targeting element and the at least one chromophore. The carrier molecule is selected so as to prevent deeper penetration into cells. At the same time, the carrier molecule must be water-compatible, must not adversely affect the specific binding capacity of the targeting element, and must not be irritating or harmful to cells. In other words, it is a physiologically and ophthalmologically acceptable polymer. Such polymers are known per se.

Suitable carrier molecules are polymers bearing functional groups through which the chromophores and optionally also targeting elements may be attached.

Any carrier molecule is suitable, provided it is water compatible in the staining complex formed, does not adversely affect the targeting function or staining, and is able to attach to chromophores and/or targeting elements. Suitable for this are water-compatible polymers such as, for example, polymers and copolymers derived from vinyl compounds, polymers and copolymers derived from polyalcohols, acrylic-based polymers and copolymers, and natural and derivatized polymers; peptides or proteins may also be considered. Examples of polymers and copolymers derived from vinyl compounds are polyvinylamine, polyvinyl alcohol, polyvinylpyrrolidone, and the corresponding copolymers. Examples of polymers and copolymers derived from polyalcohols are polyglycerols, polysaccharides, polydextrins, cyclodextrins, polyethylene glycols, celluloses, and cellulose derivatives such as hydroxypropyl methylcellulose, hydroxyethyl cellulose, hyaluronic acid, and hyaluronic acid derivatives. The acrylic-based polymers may be poly-N-(2-hydroxypropyl)methacrylamides and N-(2-hydroxypropyl)methacrylamides. Other suitable polymers are polyglutamates, polymers based on lactide and/or glycolide, e.g. polylactides, including in copolymer form, polyglutamates, polyamidoamines, and other polymers meeting the abovementioned requirements. Mixtures of the polymers mentioned may also be used.

The size of the carrier molecule may vary within a wide range and is dependent on the polymer selected in the individual case, the charge, etc. Polymers having a molecular weight in the range from 900 to 10 million may be considered. Suitable for this are carrier molecules with a molecular weight in the range from 5000 to 500 000 or 1 million, for example preferably 20 000 to 200 000, especially 30 000 to 100 000.

In one embodiment, chromophores and/or targeting elements may be attached to polymers having reactive groups. Suitable for this are, for example, homopolymers or copolymers derived from vinylamines, in which the amine groups may be attacked by, and covalently bonded to, appropriately substituted anthraquinones. The polymer for a carrier molecule may also be a peptide or protein. Particularly suitable for this embodiment are halogen-substituted anthraquinones, i.e. anthraquinones bearing a halogen group in the position designated for attachment. The halogen group should therefore be in the position at which attachment of the polymer is desired.

The advantage of using anthraquinone dyes is that reactive and less reactive positions are available for attachment on both phenyl rings and that positions which influence color (α) and positions without appreciable influence on the color of the resulting molecule (β) are available.

One embodiment accordingly uses an anthraquinone derivative that has a targeting element in at least one α-position and optionally has an attachment position for a carrier molecule in a β-position, for example a halogen such as fluorine or bromine.

Thus, in one embodiment, the tissue-targeting complex according to the invention may bear a chromogenic and targeting group in at least one α-position and be attached via a connecting group to a carrier molecule at a β-position. In a preferred embodiment, the anthraquinone derivative bears one or two targeting elements on one phenyl ring and is attached to a carrier molecule at a β-position of the other phenyl ring.

One of the functions of the carrier molecule is to prevent penetration of the staining complex into cells. This means that the carrier element should be of sufficient size to prevent uptake of the complex into a cell. On the other hand, the polymer must not be so large that it becomes insoluble and/or is no longer in dissolved form or in the form of a dispersion in water. The carrier molecule may be of any suitable shape. It may be a polymer chain to which are attached chromophores and targeting elements in any order. It may also be a branched-chain polymer in which chromophores and targeting elements are attached at the branching points. In a further embodiment, the polymer may take the form of beads, on the surface of which are attached chromophores and/or targeting elements.

Since the structure of the targeting element means it can also be attached to the chromophore, in another embodiment, the complex has a structure in which the carrier molecule has chromophore units attached to it that in turn have targeting elements attached to them.

The number of chromophores and/or targeting elements attached to a carrier molecule depends on the size of the carrier molecule, the type of monomer units, the binding capacity of the targeting element, the staining capacity of the chromophore(s). Those skilled in the art are able to use simple routine tests to establish the optimal ratio of carrier molecule/chromophore/targeting element in the individual case. The ratio of chromophore units to targeting elements is likewise noncritical and is guided, for example, by the color intensity of the chromophore, affinity of the targeting element, desired shade. It has been found that an excess of targeting elements relative to chromophores is often advantageous. A molar ratio of chromophore units to targeting elements in the range from 1:20 to 5:1, in particular 1:10 to 2:1, is considered suitable.

For the tissue-targeting complex according to the invention, only one type of chromophore is normally used. However, it is also possible to attach different chromophores to a carrier molecule or to use a mixture of carrier molecules each bearing different chromophores. All that is essential here is to achieve the desired shade in order then to stain ocular membranes.

The structure of the tissue-targeting complex according to the invention also makes it possible to provide a staining agent that appears to have different colors depending on the light source used. For example, it is possible to use a combination of tissue-targeting complexes according to the invention that have different chromophores, in which one chromophore stains at one wavelength and another chromophore becomes visible at a different wavelength of incident light.

It is also possible to use a compound with fluorescent properties as the chromophore.

The tissue-targeting complex according to the invention has a targeting element that interacts selectively with ocular membranes. It is thus possible to selectively stain ocular membranes as the target area. The invention therefore further provides for the use of the tissue-targeting complex according to the invention for staining ocular membranes.

The drawings serve to illustrate the invention, wherein

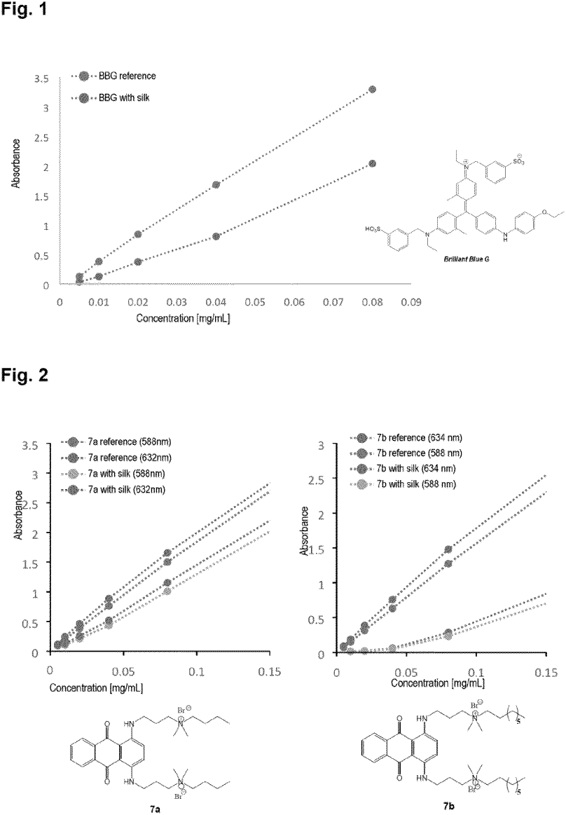

FIG. 1 shows a graphical plot of the UV/vis spectroscopy data obtained for the BBG reference (blue) and BBG MS solution (red) at concentrations of 0.005 to 0.08 mg/mL.

FIG. 2 shows a graphical plot of the UV/vis spectroscopy data obtained for the 7a and 7b references (blue and red) and 7a and 7b MS solutions (purple and green) at concentrations of 0.005 to 0.16 mg/mL.

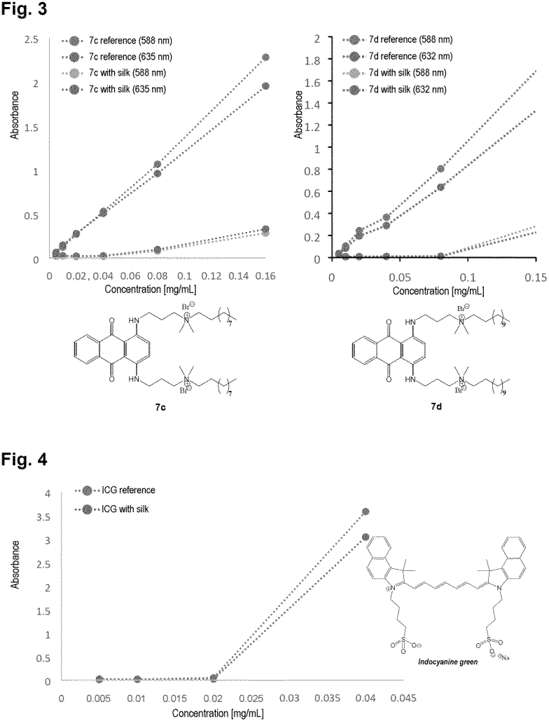

FIG. 3 shows a graphical plot of the UV/vis spectroscopy data obtained for the 7c and 7d references (blue and red) and 7c and 7d MS solutions (purple and green) at concentrations of 0.005 to 0.16 mg/mL.

FIG. 4 shows a graphical plot of the UV/vis spectroscopy data obtained for the ICG reference (blue) and ICG MS solution (red) at concentrations of 0.005 to 0.04 mg/mL.

### EXAMPLES

The invention is further illustrated by the examples below, without being limited by them. All concentrations given in the examples refer to mg of substrate per mL of total solution. Unless otherwise stated, all reactions were carried out at room temperature, i.e. approximately 25° C.

The following procedures and conditions were employed for the analyses:

### ¹H Nuclear Magnetic Resonance Spectroscopy (¹H NMR Spectroscopy)

300 MHz and 600 MHz NMR were recorded at room temperature using a Bruker Avance III FT-NMR spectrometer. Chemical shifts are reported with reference to the signal of a deuterated solvent as internal standard. Spin multiplicities have been abbreviated to s (singlet), d (doublet), dd (doublet of doublets), t (triplet) or m (multiplet).

### ¹³C Nuclear Magnetic Resonance Spectroscopy (¹³C NMR Spectroscopy)

Measurements were likewise performed on a Bruker Avance III FT-NMR spectrometer at 75 MHz and 150 MHz and at room temperature. Chemical shifts are reported with reference to the signal of a deuterated solvent as internal standard.

### MALDI-TOF-MS

Measurements were recorded using a Bruker Ultraflex TOF (time of flight) mass spectrometer. The instrument is operated using a 337 nm nitrogen laser, both in linear mode and in reflector mode. The samples were dissolved in a suitable solvent and transferred using 2-(4-hydroxyphenylazo)benzoic acid (HABA).

### ESI-MS

Measurements were recorded using a Finnigan LCQ Deca ion-trap API mass spectrometer. The substances to be determined were dissolved in a suitable solvent at a concentration of 1 mg/mL. Ionization was by electrospray ionization.

### LC-MS

Measurements were performed using an Agilent Technologies 6120 instrument with an absorption detector (214 nm) in combination with an Agilent quadrupole mass spectrometer. A 1.8 µm (2.1 × 50 mm) Agilent SB-C₁₈ column was used, with H₂O:MeCN + 0.1% formic acid as solvent mixture at room temperature. The flow rate was 0.4 mL/min with a solvent mixture of 95:5 H₂O:MeCN and 5:95 H₂O:MeCN.

### GC-MS

Measurements were recorded using a Thermo Finnigan Trace DSQ (dual-stage quadrupole). Ionization of the samples was by electron impact ionization (EI).

### FTIR Spectroscopy

FTIR spectra were recorded at room temperature using a Nicolet 6700 FTIR spectrometer with a LOT ATR attachment. The wavenumber scale was calibrated using a HeNe laser. Measurements were recorded over a 4000-300 cm⁻¹ range.

### UV/VIS Spectroscopy

UV/vis spectra were recorded using an Analytik Jena AG Specord® 210 Plus double-beam spectrometer. Measurements were performed at room temperature between 300 and 800 nm using a quartz glass cuvette with a path length of 1 cm. Evaluation was with the aid of Analytik Jena AG WinAspect Plus software.

### Thin-Layer Chromatography

Thin-layer chromatography was carried out using Merck plates (silica gel 60 F₂₅₄ on aluminum foil).

### HPLC

RP-HPLC (reversed-phase high performance liquid chromatography) measurements were recorded using an Agilent 1200 with absorption and fluorescence detector. An Agilent Zorbax Eclipse XDB-C₁₈ column (4.6 × 100 mm) at a flow rate of 1 mL/min at 60° C. was used. A binary eluent mixture of H₂O and MeCN containing 0.1% trifluoroacetic acid was employed. The measurements were performed by gradient elution over a period of 10 min from acetonitrile/water 5:95 to 95:5.

### Example 1

### Synthesis of 1,4-difluoroanthraquinone (3)

13.4 g (0.9 mol) of phthalic anhydride (1), 48.8 g (0.37 mol) of AlCl₃, and 108 mL (2 mol) of 1,4-difluorobenzene (2) are heated under reflux for 48 h. The excess 1,4-difluorobenzene is then recovered by distillation. To the brown residue is added 400 mL of 1 N hydrochloric acid and the residue is extracted with three 600 mL portions of chloroform. The combined organic phases are concentrated under reduced pressure to approximately 50 mL. The intermediate product is then precipitated with at least 100 mL of low-boiling petroleum ether. The brownish solid is filtered off and then dried. (Yield: 66.7% of theory.) The solid is placed in 200 g of polyphosphoric acid and heated at 140° C. for 2 h. After cooling to room temperature, the viscous reaction mixture is added to 1 L of ice-water and extracted with three 400 mL portions of dichloromethane. The organic phase is concentrated under reduced pressure and the product (3) can then be isolated by column chromatography purification on silica gel (hexane: ethyl acetate [7:1]).

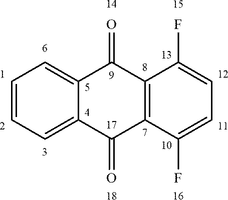 [SMILES: O=C1c2ccccc2C(=O)c2c(F)ccc(F)c21]

Yield: 13.2 g (54.1 mmol) 60.1% of theory. (literature = 50%)

FTIR (diamond): v= 3084 (s, υ_{c=c}), 1678 (s, υ_{c=o}), 1587 (m, υ_{c=c}), 1249 (vs, υ_{C-F}), 719 (s, δ_{Ar-H}) cm⁻¹.

¹H NMR (300 MHz, CDCl₃, RT): δ = 8.20-8.29 (m, 2H, Ar—H^{3,6}), 7.76-7.85 (m, 2H, Ar—H^{1,2}), 7.43-7.53 (m, 2H, Ar—H^{11,12}) ppm.

¹³C NMR (300 MHz, DMSO-d₆, RT): δ= 180.23 (C^{9,17}), (158.55, 155.10) (C^{10,13}), 134.40 (C^{1,2}), 133.04 (C^{4,5}), 126.23 (C^{3,6}), 124.6-125.25 (C^{7,8}), 121.43-121.60 (C^{11,12}) ppm.

EI-MS m/z: 244 [M]

### Example 2

### Synthesis of 1,4-bis[[2-(dimethylamino)propyl]amino]anthraquinone (5)

3.0 g (12.3 mmol) of 1,4-difluoroanthraquinone (3) is heated at approximately 100° C. with 3.1 mL (24.6 mmol) of N,N-dimethylaminopropylamine (4) in 25 mL of DMSO under a nitrogen atmosphere. After stirring for 24 h, the reaction mixture is cooled to room temperature and diluted with water. The blue crude product is extracted with CHCl₃, concentrated under reduced pressure, and purified by column chromatography on silica gel (CH₂Cl₂:MeOH:NEt₃ [3:4:0.1]).

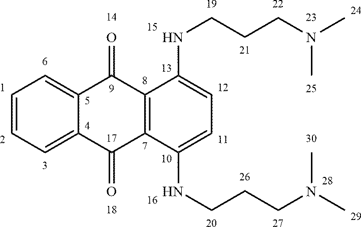 [SMILES: CN(C)CCCNc1ccc(NCCCN(C)C)c2c1C(=O)c1ccccc1C2=O]

Yield: 2.1 g (5.2 mmol) 42.3% of theory.

FTIR (diamond): v= 3425 (w, υ_{NH}), 3068 (w, υ_{Ar-H}), 2948 (m, υ_{CH2}), 2859 (m, υ_{CH2}), 2787 (m, υ)_{(N-CH3)C-H}), 2766 (m, υ(_{N-CH3})_{N-C}), 1642 (w, υ_{c=o}), 1591 (m, υ_{c=c}), 1567 (m, υ_{c=c}), 1556 (s, υ_{c=c}), 1457 (m, δ_{CH2,CH3}), 731 (s, υ_{Ar-H}) cm⁻¹.

¹H NMR (300 MHz, CDCl₃, RT): δ = 10.83 (t, ³J_{H,H}=5.35 Hz, 2H, NH^{15,16}), 8.34 (dd, ³J= 5.92, ⁴J= 3.27 Hz, 2H, Ar—H^{3,6}), 7.69 (dd, ³J= 5.91, ⁴J= 3.31 Hz, 2H, Ar—H^{1,2}), 7.30 (s, 2H, Ar—H^{11,12}), 3.48 (m, 4H, CH₂^{19,20}), 2.45 (t, ³J=6.94 Hz, 4H, CH₂^{22,27}), 2.27 (s, 12H, NMe₂^{24,24,29,30}), 1.92 (t, ³J= 7.1 Hz, 4H, CH₂^{21,26}) ppm.

¹³C NMR (75 MHz, DMSO-d₆, RT): δ= 182.41 (C^{9,17}), 146.42 (C^{10,13}), 134.70 (C^{1,2}), 132.07 (C^{4,5}), 126.12 (C^{3,6}), 123.80 (C^{7,8}), 109.85 (C^{11,12}), 57.78 (C^{22,27}), 45.67 (C^{24,25,29,30}), 41.03 (C^{19,20}), 28.05 (C^{21,26}) ppm.

ESI-MS m/z: 205.3 [M+2H]²⁺

Elemental analysis: theoretical values: C: 70.56; H: 7.90; N: 13.71
analysis results: C: 70.65; H: 7.70; N: 13.58

### Example 3

### Synthesis of N,N′=(((9,10-dioxo-9,10-dihydroanthracene-1,4-diyl)bis(azanediyl))bis(propane-3,1-diy1))bis(N,N-dimethylbutan-1-aminium) bromide (7a)

0.5 g (1.23 mmol) of 1,4-bis[[2-(dimethylamino)propyl]amino]anthraquinone (5) is heated at reflux with 1.68 g (1.3 mL, 12.25 mmol) of 1-bromobutane (6a) in approximately 5 mL of acetone for 24 h. After cooling the solution to room temperature, the product is completely precipitated in hexane, filtered off, and washed with hexane. The blue solid is dried in high vacuum.

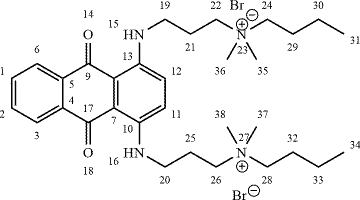

Yield: 0.81 g (1.19 mmol) 97% of theory.

FT-IR (diamond): v= 3404 (br, υ_{NH}), 3006 (w, υ_{Ar-H}), 2958 (m, υ_{CH2}), 2871 (m, υ_{CH2}), 1641 (w, υ_{c=o}), 1607 (w, υ_{c=c}), 1594 (m, υ_{c=c}), 1572 (s, υ_{c=c}), 1519 (m, υ_{c=c}), 1467 (m, δ_{CH2,CH3}), 724 (s, υ_{Ar-H}) cm⁻¹.

¹H NMR (300 MHz, DMSO-d₆, RT): δ[ppm]= 10.79 (t, ³J= 5.82 Hz, 2H, NH^{15,16}), 8.24 (dd, ³J= 5.85 Hz, ⁴J= 3.4 Hz, 2H, Ar—H^{3,6}), 7.83 (m, 2H, Ar—H^{1,2}), 7.57 (s, 2H, Ar—H^{11,12}), 3.55 (m, 4H, CH₂^{19,20}), 3.44 (m, 4H, CH₂^{22,26}), 3.31 (m, 4H, CH₂^{24,28}), 3.07 (s, 12H, NMe₂³⁵⁻³⁸), 2.09 (m, 4H, CH₂^{21,25}), 1.64 (m, 4H, CH₂^{29,32}), 1.29 (m, 4H, CH₂^{30,33}), 0.91 (t, ³J= 7.3 Hz 6H, CH₃^{31,34}) ppm.

¹³C NMR (75 MHz, DMSO-d₆, RT): δ= 181.12 (C^{9,17}), 145.63 (C^{10,13}), 133.72 (C^{1,2}), 132.55 (C^{4,5}), 125.67 (C^{3,6}), 124.52 (C^{7,8}), 108.84 (C^{11,12}), 62.89 (C^{24,28}), 60.62 (C^{22,26}), 50.24 (C^{35,36,37,38}), 39.5 (C^{19,20} under the DMSO-d₆ signal), 23.67 (C^{29,32}), 22.73 (C^{21,25}), 19.13 (C^{30,33}), 13.40 (C^{31,34}) ppm.

LC-MS m/z: 261.2 [M-2Br]²⁺

HPLC: 97.07% 6.03 min

### Example 4

### Synthesis of N,N′-(((9,10-dioxo-9,10-dihydroanthracene-1,4-diyl)bis(azanediyl))bis(propane-3,1-diyl))bis(N,N-dimethyloctan-1-aminium) bromide (7b)

0.65 g (1.33 mmol) of 1,4-bis[[2-(dimethylamino)propyl]amino]anthraquinone (5) is heated at reflux with 2.6 g (2.3 mL, 13.3 mmol) of 1-bromooctane (6b) in approximately 5 mL of acetone for 24 h. After cooling the solution to room temperature, the product is completely precipitated in hexane, filtered off, and washed with hexane. The blue solid is dried in high vacuum.

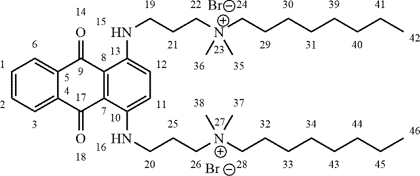 [SMILES: [Br-] ; CCCCCCCC[N+](C)(C)CCCNc1ccc(NCCC[N+](C)(C)CCCCCCCC)c2c1C(=O)c1ccccc1C2=O ; [Br-]]

Yield: 0.99 g (1.25 mmol) 94% of theory.

FT-IR (diamond): v= 3419 (br, υ_{NH}), 3004 (w, υ_{Ar-H}), 2953 (m, υ_{CH2}), 2923 (m, υ_{CH2}), 2854 (m, υ_{CH2}), 1645 (w, υ_{c=o}), 1595 (w, υ_{c=c}), 1576 (m, υ_{c=c}), 1588 (s, υ_{c=c}), 1467 (m, δ_{CH2,CH3}), 717 (s, υ_{Ar-H}) cm⁻¹.

¹H NMR (300 MHz, DMSO-d₆, RT): δ[ppm]= 10.79 (t, ³J= 5.84 Hz, 2H, NH^{15,16}), 8.25 (dd, ³J= 5.90 Hz, 4J=3.30 Hz, 2H, Ar—H^{3,6}), 7.82 (m, 2H, Ar—H^{1,2}), 7.57 (s, 2H, Ar—H^{11,12}), 3.55 (m, 4H, CH₂^{19,20}), 3.43 (m, 4H, CH₂^{22,26}), 3.29 (m, 4H, CH₂^{24,28}), 3.06 (s, 12H, NMe₂³⁵⁻³⁸), 2.09 (m, 4H, CH₂^{21,25}), 1.64 (m, 4H, CH₂^{29,32}), 1.12-1.31 (m, 20H, CH₂^{30,31,33,34,39-41,43-45}), 0.82 (m, 6H, CH₃^{42,46}) ppm.

¹³C NMR (75 MHz, DMSO-d₆, RT): δ= 181.10 (C^{9,17}), 145.63 (C^{10,13}), 133.74 (C^{1,2}), 132.56 (C^{4,5}), 125.71 (C^{3,6}), 124.56 (C^{7,8}), 108.84 (C^{11,12}), 62.85 (C^{24,28}), 60.38 (C^{22,26}), 50.32 (C³⁵⁻³⁸), 39.5 (C^{19,20} under the DMSO-d₆ signal), 31.16 (C^{40,44}), 28.50 (C^{31,34,39,43}), 25.80 (C^{30,33}), 22.73 (_{C}^{29,32}), 22.02 (C^{21,25}), 21.71 (C^{41,45}), 13.94 (C^{42,46}) ppm.

LC-MS m/z: 317.3 [M-2Br]²⁺

HPLC: 96.98% 7.75 min

### Example 5

### Synthesis of N,N′=(((9,10-dioxo-9,10-dihydroanthracene-1,4-diyl)bis(azanediyl))bis(propane-3,1-diyl))bis(N,N-dimethyldecan-1-aminium) Bromide (7c)

0.33 g (0.81 mmol) of 1,4-bis[[2-(dimethylamino)propyl]amino]anthraquinone (5) is heated at reflux with 1.69 mL (8.1 mmol) of 1-bromodecane (6c) in 5 mL of acetone for approximately 48 h. After cooling the solution to room temperature, the product is completely precipitated in hexane, filtered off, and washed with hexane. The blue solid is dried in high vacuum.

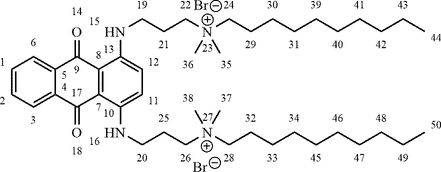 [SMILES: [Br-] ; CCCCCCCCCC[N+](C)(C)CCCNc1ccc(NCCC[N+](C)(C)CCCCCCCCCC)c2c1C(=O)c1ccccc1C2=O ; [Br-]]

Yield: 0.68 g (0.8 mmol) 99% of theory.

FT-IR (diamond): v= 3419 (br, υ_{NH}), 3007 (w, υ_{Ar-H}), 2948 (m, υ_{CH2}), 2920 (m, υ_{CH2}), 2852 (m, υ_{CH2}), 1639 (w, υ_{c=o}), 1595 (m, υ_{c=c}), 1579 (m, υ_{c=c}), 1562 (s, υ_{c=c}), 1467 (m, δ_{CH2,CH3}), 731 (s, υ_{Ar-H}) cm⁻¹.

¹H NMR (300 MHz, DMSO-d₆, RT): δ= 10.79 (t, ³J= 5.73 Hz, 2H, NH^{15,16}), 8.24 (dd, ³J= 5.88 Hz, ⁴J= 3.31 Hz, 2H, Ar—H^{3,6}), 7.82 (m, 2H, Ar—H^{1,2}), 7.57 (s, 2H, Ar—H^{11,12}), 3.55 (m, 4H, CH₂^{19,20}), 3.43 (m, 4H, CH₂^{22,26}), 3.29 (m, 4H, CH₂^{24,28}), 3.06 (s, 12H, NMe₂³⁵⁻³⁸), 2.08 (m, 4H, CH₂^{21,25}), 1.63 (m, 4H, CH₂^{29,32}), 1.12-1.32 (m, 28H, CH₂^{30,31,33,34,39-43,45-49}), 0.83 (m, 6H, CH₃^{44,50}) ppm.

¹³C NMR (75 MHz, DMSO-d₆, RT): δ= 181.17 (C^{9,17}), 145.70 (C^{10,13}), 133.79 (C^{1,2}), 132.62 (C^{4,5}), 125.77 (C^{3,6}), 124.62 (C^{7,8}), 108.90 (C^{11,12}), 62.87 (C^{24,28}), 60.41 (C^{22,26}), 50.39 (C³⁵⁻³⁸), 39.5 (C^{19,20} under the DMSO-d₆ signal), 31.31 (C^{42,48}), 28.57-28.94 (C^{31,34,39}-^{41,45}-⁴⁷), 25.82 (C^{30,33}), 22.75 (C^{29,32}), 22.11 (C^{21,25}), 21.74 (C^{43,49}), 13.98 (C^{44,50}) ppm.

LC-MS m/z: 345.4 [M-2Br]²⁺

HPLC: 96.92% 8.57 min

Elemental analysis: theoretical values: C: 62.11; H: 8.77; N: 6.58
analysis results: C: 61.87; H: 8.72; N: 6.42

### Example 6

### Synthesis of N,N′(((9,10-dioxo-9,10-dihydroanthracene-1,4-diyl)bis(azanediyl))bis(propane-3,1-diyl))bis(N,N-dimethyldodecan-1-aminium) Bromide (7d)

0.35 g (0.86 mmol) of 1,4-bis[[2-(dimethylamino)propyl]amino]anthraquinone (5) is heated at reflux with 0.85 g (0.89 mL, 3.43 mmol) of 1-bromododecane (6d) in approximately 2 mL of acetone for 24 h. After cooling the solution to room temperature, the product is completely precipitated in hexane, filtered off, and washed with hexane. The blue solid is dried in high vacuum.

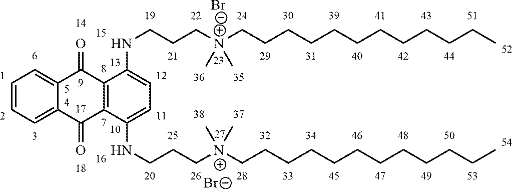 [SMILES: [Br-] ; CCCCCCCCCCCC[N+](C)(C)CCCNc1ccc(NCCC[N+](C)(C)CCCCCCCCCCCC)c2c1C(=O)c1ccccc1C2=O ; [Br-]]

Yield: 0.71 g (0.79 mmol) 91% of theory.

FT-IR (diamond): v= 3425 (br, υ_{NH}), 3006 (w, υ_{Ar-H}), 2948 (m, υ_{CH2}), 2919 (s, υ_{CH2}), 2851 (m, υ_{CH2}), 1639 (w, υ_{c=o}), 1595 (m, υ_{c=c}), 1580 (m, υ_{c=c}), 1562 (s, υ_{c=c}), 1468 (m, δ_{CH2,CH3}), 731 (s, υ_{Ar-H}) cm⁻¹.

¹H NMR (300 MHz, DMSO-d₆, RT): δ= 10.78 (m, 2H, NH^{15,16}), 8.25 (m, 2H, Ar—H^{3,6}), 7.82 (m, 2H, Ar—H^{1,2}), 7.56 (s, 2H, Ar—H^{11,12}), 3.55 (m, 4H, CH₂^{19,20}), 3.21-3.46 (m, CH₂^{22,26,24,28} obscured by the H₂O signal), 3.05 (s, 12H, NMe₂³⁵⁻³⁸), 2.08 (m, 4H, CH₂^{21,25}), 1.63 (m, 4H, CH₂^{29,32}), 1.11-1.32 (m, 36H, CH₂^{30,31,33,34,39-51,53}), 0.84 (m, 6H, CH₃^{52,54}) ppm.

¹³C NMR (75 MHz, DMSO-d₆, RT): δ= 181.17 (C^{9,17}), 145.65 (C^{10,13}), 133.77 (C^{1,2}), 132.61 (C^{4,5}), 125.74 (C^{3,6}), 124.54 (C^{7,8}), 108.89 (C^{11,12}), 62.80 (C^{24,28}), 60.38 (C^{22,26}), 50.38 (C³⁵⁻³⁸), 39.5 (C^{19,20} under the DMSO-d₆ signal), 31.29 (C^{44,50}), 28.53-29.1 (C^{31,34,39-43,45-49}), 25.78 (C^{30,33}), 22.70 (C^{29,32}), 22.09 (C^{21,25}), 21.69 (C^{51,53}), 13.96 (C^{52,54}) ppm.

LC-MS m/z: 373.4 [M-2Br]²⁺

HPLC: 96.44% 9.79 min

### Example 7

An ILM model substrate that is similar to the ILM was used to test the binding capacity of known dyes and of staining complexes according to the invention. Silk granules have been found to show good suitability as an ILM model substrate. To examine the binding of various dyes to the ILM model substrate, UV/vis measurements of the dyes Brilliant Blue G (BBG), compounds 7a-d, and Indocyanine green (ICG) were performed at various concentrations both before and after treatment with the model substrate. The aim of these measurements is to compare the binding strength of the individual dyes. The results are shown below and are each depicted in graph form in FIGS. 1 to 4.

Stock solutions of the dyes with a concentration of 5 mg/mL were prepared using phosphate-buffered saline (PBS) at physiological pH 7.4. Because of the poor solubility of ICG in saline solutions, double-distilled water was used. The concentrations prepared are shown in the Table.

TABLE 1

Preparation of dye solutions for the UV/vis-monitored study of binding to ILM model substrate

| Concentration [mg/mL] | Composition of dye solution |
|---|---|
| 0.005 | 6000 µL PBS + 6 µL stock solution |
| 0.01 | 5994 µL PBS + 12 µL stock solution |
| 0.02 | 5982 µL PBS + 24 µL stock solution |
| 0.04 | 5958 µL PBS + 48 µL stock solution |
| 0.08 | 5910 µL PBS + 96 µL stock solution |
| 0.16 | 5814 µL PBS + 192 µL stock solution |

To ensure measurement is as accurate as possible, the dye-model substrate solutions and the references were not prepared separately, but likewise used as stock solutions. This was done by preparing two and a half times the amount required as shown in Table 1.

Subsequently, 6 mg of ILM model substrate plus 6 mL of dye solution and a reference solution comprising only 6 mL of dye solution were stirred for exactly 10 minutes and then centrifuged together for 8 minutes at 6000 rpm. The solutions were photographed and the carefully decanted supernatants analyzed by UV/vis spectroscopy. The ratios of the absorption maxima λₘₐₓ with the model substrate to the absorption maximum λₘₐₓ of the reference were calculated. This allowed the binding strengths of the individual dyes to be compared. Graphical plots of the results are shown in FIG. 1. The tabulated summary of the data obtained is shown below.

### A) UV/Vis Measurements of the Vital Dye Brilliant Blue G (BBG) (Comparison)

As previously explained, Brilliant Blue G is a dye commonly used in chromovitrectomy which selectively stains the inner limiting membrane. This means that good staining and a consequent pronounced decrease in its absorption maximum after treatment with a model substrate would also be expected in binding studies with a model substrate.

As can be demonstrated by FIG. 1, the expected good binding to the model substrate is confirmed. Only the strongest absorption maximum, which is responsible for imparting the blue color, was considered here. The maximum of the references is at 585 nm here. The samples treated with the model substrate show a slight shift in the maximum which, as the concentration increases, approaches that of the reference. The absorption of the references is considerably more intense than that of the samples treated with the model substrate. This suggests a good interaction between the dye and the model substrate. Table 2 shows the absorption maxima of each dye solution. The ratio of λₘₐₓ with the model substrate to λₘₐₓ of the associated reference is confirmed by the graphical plot of the binding strength. At a concentration of 0.005 mg/mL, the sample treated with model substrate shows only 24% of the reference maximum. This equates to 76% binding of the dye. Binding decreases as the concentration of the solution increases. At a concentration of 0.08 mg/mL, binding of only 48% of the dye can be demonstrated.

TABLE 2

UV/vis spectroscopy data obtained for the BBG reference and BBG model substrate (MS) solution at concentrations of 0.005 to 0.08 mg/mL

| Concentration [mg/mL] | λₘₐₓ Reference (585 nm) | λₘₐₓ With MS (x nm) | Ratio λₘₐₓ with MS and reference |
|---|---|---|---|
| 0.005 | 0.1241 | 0.0299 (603 nm) | 24% |
| 0.01 | 0.3755 | 0.1217 (595 nm) | 32% |
| 0.02 | 0.835 | 0.3672 (589 nm) | 44% |
| 0.04 | 1.6741 | 0.8006 (587 nm) | 48% |
| 0.08 | 3.2859 | 2.0313 (585 nm) | 62% |

### B) UV/Vis Measurements of Anthraquinone Dyes 7a-d (According to the Invention)

The anthraquinone dyes 7a-d according to the invention were likewise tested in respect of their strength of binding to the model substrate. Because of the lower absorption maximum compared with the known BBG, 6 concentrations from 0.005 mg/mL to 0.16 mg/mL were measured in this case. Graphical plots of the UV/vis spectroscopy data obtained are shown in FIGS. 2 and 3.

FIGS. 2 and 3 demonstrate clearly the very good binding of anthraquinone dyes 7b-d to the model substrate. Since the anthraquinone dyes have two absorption maxima, the graphs show two reference lines and two sample lines. The absorption maxima are at 588 nm and 632-635 nm. The reference absorption maxima here are well above those of the values treated with model substrate. Higher concentrations could not be recorded, because the absorption of the reference was too high. Table 3 shows the ratios of the λₘₐₓ values of the MS samples in relation to the corresponding references. The exact measured data obtained for the UV/vis spectroscopy investigations are shown in Tables 4 to 7. As the length of the alkyl radicals increases, the interaction of the dyes with the model substrate is heightened. Whereas compound 7a with a butyl radical has λₘₐₓ ratios only of between approximately 50 to 80%, improved values can be achieved with dyes 7b-d. What is however clear is that, as the length of the alkyl radical increases, the extinction coefficient of the dye also decreases. This is a consequence both of the molecular weight of the compound and of the increasing hydrophobic character.

TABLE 3

UV/vis spectroscopy data obtained for the 7 references and 7 MS solution at concentrations of 0.005 to 0.16 mg/mL

| Concentration [mg/mL] | 7a | | 7b | | 7c | | 7d | |
|---|---|---|---|---|---|---|---|---|
| | | | Ratio λₘₐₓ with MS and reference | | | | | |
| | 588 nm | 632 nm | 588 nm | 634 nm | 588 nm | 635 nm | 588 nm | 634 nm |
| 0.005 | 54% | 54% | 11% | 9% | 48% | 37% | 12% | 13% |
| 0.01 | 52% | 52% | 10% | 8% | 21% | 17% | 7% | 8% |
| 0.02 | 54% | 55% | 6% | 6% | 7% | 7% | 3% | 4% |
| 0.04 | 57% | 58% | 8% | 8% | 4% | 5% | 3% | 3% |
| 0.08 | 67% | 69% | 18% | 19% | 8% | 10% | 1% | 2% |
| 0.16 | 76% | 78% | 32% | 34% | 13% | 17% | 18% | 18% |

Table 4: UV/vis spectroscopy data obtained for the 7a references and 7a MS solutions at concentrations of 0.005 to 0.16 mg/mL

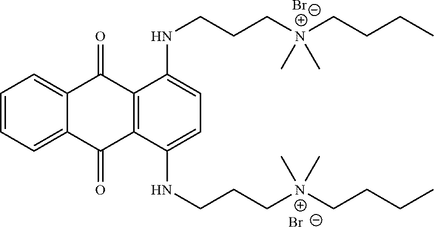

| Concentration [mg/mL] | λₘₐₓ Reference (588 nm) | λₘₐₓ With MS (588 nm) | λₘₐₓ Reference (632 nm) | λₘₐₓ With MS (632 nm) | Ratio λₘₐₓ with MS and reference 588 nm 632 nm | |
|---|---|---|---|---|---|---|
| 0.005 | 0.0932 | 0.0502 | 0.1159 | 0.0624 | 54% | 54% |
| 0.01 | 0.2000 | 0.1032 | 0.2448 | 0.1268 | 52% | 52% |
| 0.02 | 0.3868 | 0.2103 | 0.4640 | 0.2560 | 54% | 55% |
| 0.04 | 0.7611 | 0.4333 | 0.8862 | 0.5177 | 57% | 58% |
| 0.08 | 1.500 | 1.0086 | 1.6571 | 1.1524 | 67% | 69% |
| 0.16 | 2.8594 | 2.1625 | 2.9961 | 2.3477 | 76% | 78% |

Table 5: UV/vis spectroscopy data obtained for the 7b references and 7b MS solutions at concentrations of 0.005 to 0.16 mg/mL

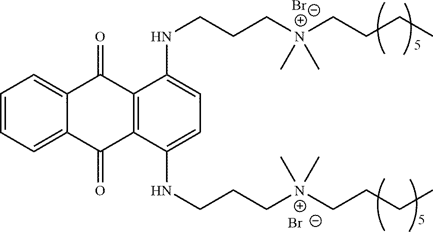

| Concentration [mg/mL] | λₘₐₓ Reference (588 nm) | λₘₐₓ With MS (588 nm) | λₘₐₓ Reference (634 nm) | λₘₐₓ With MS (634 nm) | Ratio λₘₐₓ with MS and reference 588 nm 634 nm | |
|---|---|---|---|---|---|---|
| 0.005 | 0.0733 | 0.0078 | 0.0930 | 0.0082 | 11% | 9% |
| 0.01 | 0.1508 | 0.0150 | 0.1890 | 0.0159 | 10% | 8% |
| 0.02 | 0.3142 | 0.0201 | 0.3881 | 0.0232 | 6% | 6% |
| 0.04 | 0.6307 | 0.0496 | 0.7606 | 0.0609 | 8% | 8% |
| 0.08 | 1.2728 | 0.2326 | 1.4791 | 0.2873 | 18% | 19% |
| 0.16 | 2.4402 | 0.7719 | 2.6985 | 0.9204 | 32% | 34% |

Table 6: UV/vis spectroscopy data obtained for the 7c references and 7c MS solutions at concentrations of 0.005 to 0.16 mg/mL

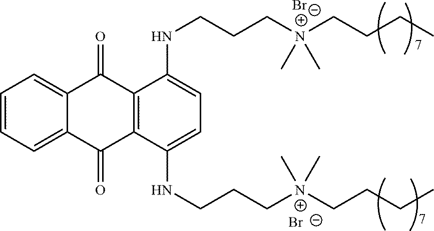

| Concentration [mg/mL] | λₘₐₓ Reference (588 nm) | λₘₐₓ With MS (588 nm) | λₘₐₓ Reference (635 nm) | λₘₐₓ With MS (635 nm) | Ratio λₘₐₓ with MS and reference 588 nm 635 nm | |
|---|---|---|---|---|---|---|
| 0.005 | 0.0503 | 0.0243 | 0.0633 | 0.0234 | 48% | 37% |
| 0.01 | 0.124 | 0.0267 | 0.1476 | 0.0261 | 21% | 17% |
| 0.02 | 0.2708 | 0.0205 | 0.2786 | 0.0216 | 7% | 7% |
| 0.04 | 0.5298 | 0.0218 | 0.5068 | 0.0257 | 4% | 5% |
| 0.08 | 1.0636 | 0.0799 | 0.9575 | 0.0977 | 8% | 10% |
| 0.16 | 2.2754 | 0.2842 | 1.9492 | 0.3300 | 13% | 17% |

Table 7: UV/vis spectroscopy data obtained for the 7d references and 7d MS solutions at concentrations of 0.005 to 0.16 mg/mL

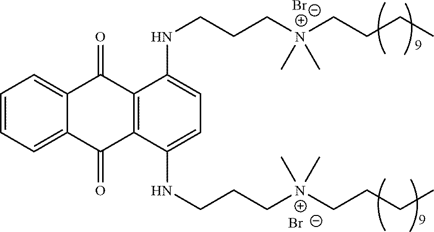

| Concentration [mg/mL] | λₘₐₓ Reference (588 nm) | λₘₐₓ With MS (588 nm) | λₘₐₓ Reference (634 nm) | λₘₐₓ With MS (634 nm) | Ratio λₘₐₓ with MS and reference 588 nm 634 nm | |
|---|---|---|---|---|---|---|
| 0.005 | 0.0369 | 0.0043 | 0.0304 | 0.0041 | 12% | 13% |
| 0.01 | 0.1045 | 0.0074 | 0.0845 | 0.0071 | 7% | 8% |
| 0.02 | 0.2440 | 0.0081 | 0.1949 | 0.0080 | 3% | 4% |
| 0.04 | 0.3649 | 0.0101 | 0.2895 | 0.0100 | 3% | 3% |
| 0.08 | 0.8039 | 0.0120 | 0.6368 | 0.0120 | 1% | 2% |
| 0.16 | 1.8155 | 0.3218 | 1.4306 | 0.2580 | 18% | 18% |

### C) UV/Vis Measurements of the Vital Dye Indocyanine Green (ICG)

Indocyanine green is one of the most important dyes in chromovitrectomy, particularly in the field of ILM peeling. The dye does, however, give rise to some problems and also to handling difficulties. Although it has long since been used for staining the inner limiting membrane and epiretinal membranes, studies have shown the dye to have numerous disadvantages. In addition to postoperative deterioration of vision, low stability in the presence of light is a serious problem when working with the dye. Moreover, ICG cannot be measured or used in phosphate-buffered saline, as this causes the compound to precipitate. This means that the subsequent measurements also had to be performed in double-distilled water, since measurements in buffer solution showed only settling-out of the dye after centrifugation. Graphical plots of the UV/vis data obtained are shown in FIG. 4.

As is clear from the data presented, ICG showed some problems in the present measurements too. Whereas the dye showed virtually no absorption up to a concentration of 0.02 mg/mL, a concentration of 0.04 mg/mL was sufficient for Indocyanine green to attain an absorption of 3.5822 and thus the maximum possible measured value. Use of a higher concentration was not possible, because of the excessively high absorption and consequent inaccurate measurement. As can be seen from Table 8, although the ratios of the λₘₐₓ values of the reference and of the model substrate-dye solution revealed positive binding of the dye to the model substrate, the results are subject to substantial variation within the margin of error due to the low concentration. This may be a consequence both of the weak absorption at low concentrations and of photolytic decomposition. During the measurement method performed, it was not possible to ensure the complete exclusion of light.

TABLE 8

UV/vis spectroscopy data obtained for the ICG reference and ICG MS solution at concentrations of 0.005 to 0.04 mg/mL

| Concentration [mg/mL] | λₘₐₓ Reference (779 nm) | λₘₐₓ With MS (780 nm) | Ratio λₘₐₓ with MS and reference |
|---|---|---|---|
| 0.005 | 0.0226 | 0.0106 | 47% |
| 0.01 | 0.0162 | 0.0154 | 95% |
| 0.02 | 0.0477 | 0.0204 | 42% |
| 0.04 | 3.5822 | 3.0389 | 85% |

### Example 8

Some example syntheses and schemes for the preparation of the dyes are shown below. The 6-methyl-substituted derivative of 1,4-difluoroanthraquinone was used, which was synthesized from 4-methylphthalic anhydride and 1,4-difluorobenzene and then had to undergo oxidation. This method permits modification of the side groups without potential side reactions due to the acid. It also affords the possibility of a simplified purification. Subsequent oxidation of the methyl group in the 6-position should result in the desired acid group. The synthesis of 1,4-difluoro-6-methylanthraquinone (14) is shown in Scheme 1.

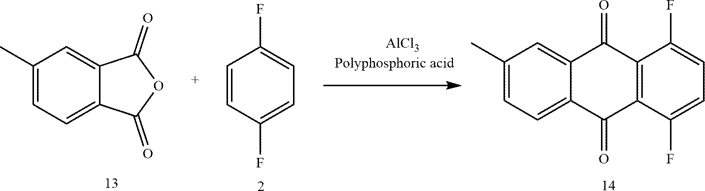

Scheme 1: Synthesis of 1,4-difluoro-6-methylanthraquinone (14)

The reaction was carried out in analogous manner to the synthesis of 1,4-difluoroanthraquinone (1). After purification by column chromatography, the successful synthesis of compound 14 in low yields of around 20% was verified by 1H NMR, 13C NMR, and IR spectroscopy, and by elemental analysis. This was followed by modification with N,N-dimethylaminopropylamine (4) to give the difunctionalized product 1,4-bis((3-(dimethylamino)propyl)amino)-6-methylanthraquinone (15) (Scheme 2). After purification of the product mixture by column chromatography, compound 15 was verified unambiguously by 1H NMR. In this case too, the protons vicinal to fluorine give rise to a singlet after successful conversion to dye 15.

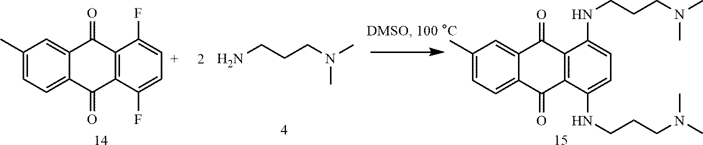

Scheme 2: Reaction of 1,4-difluoro-6-methylanthraquinone (14) with N,N-dimethylaminopropylamine (4) to dye 10

Because the higher selectivity of the quaternary anthraquinone dye having a C10-substituent was already known at this time, quaternization of the doubly modified 1,4-difluoro-6-methylanthraquinone 15 was carried out only with 1-bromodecane. Scheme 3 shows the reaction carried out.

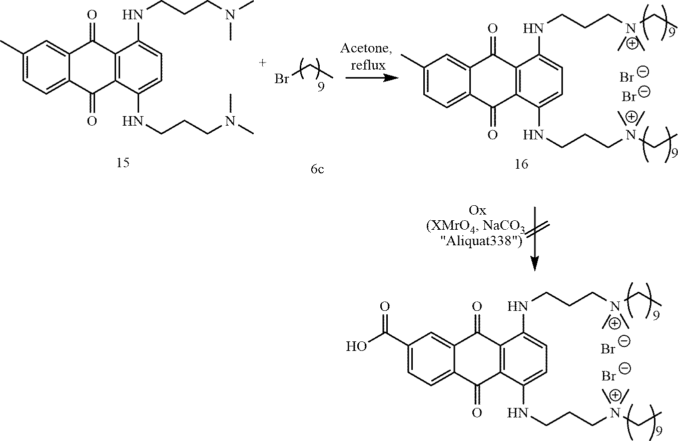

Scheme 3: Reaction of the difunctionalized 1,4-difluoro-6-methylanthraquinone (15) with 1-bromodecane (6c)

The successful synthesis of anthraquinone dye 16 was verified unambiguously by¹H NMR. The success of the synthesis is indicated by the shift of the signals for the amine-bound methyl groups from 2.21 ppm to 3.04 ppm. Subsequent oxidation of the methyl group in compound 16 by potassium permanganate with sodium carbonate and the phase-transfer catalyst “Aliquat 336” in dichloromethane/water (1:1) was unsuccessful. A further attempt to obtain the carboxyl-modified anthraquinone compound was accordingly made directly from trimellitic anhydride (17) and 1,4-difluorobenzene (1). The reaction carried out is shown in Scheme 4. 5,8-Difluoro-9,10-dioxo-9,10-dihydroanthracene-2-carboxylic acid (18) was verified by 1H NMR spectroscopy and by mass spectrometry.

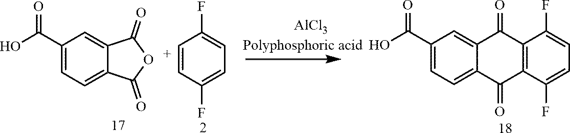

Scheme 4: Synthesis of the dye 5,8-difluoro-9,10-dioxo-9,10-dihydroanthracene-2-carboxylic acid, modified with an acid group in the 6-position (18)

The further conversion to 19 was carried out in analogous manner to the earlier approach by reaction with N,N-dimethylaminopropylamine (4) and is shown in Scheme 5.

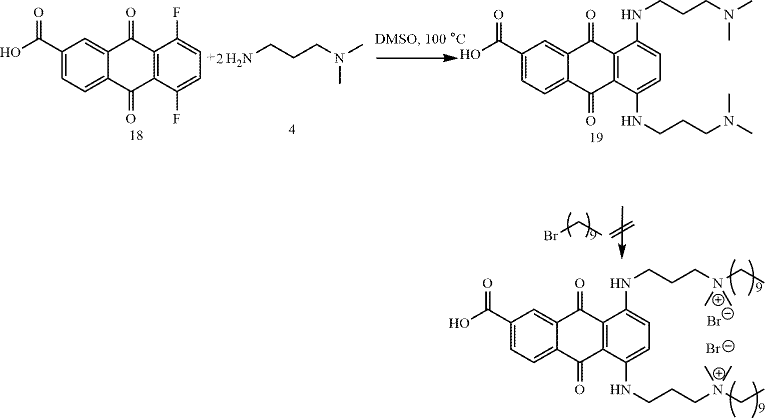

Scheme 5: Reaction of the carboxyl-modified 1,4-difluoroanthraquinone (18) with N,N-dimethylaminopropylamine (4) to give 5,8-bis((3-(dimethylamino)propyl)amino)-9,10-dioxo-9,10-dihydroanthracene-2-carboxylic acid

The successful synthesis of the anthraquinone dye 5,8-bis((3-(dimethylamino)propyl)amino)-9,10-dioxo-9,10-dihydroanthracene-2-carboxylic acid (19) is confirmed by 1H NMR spectroscopy and mass spectrometry analyses. The final step to incorporate the positive charge by reaction with 1-bromodecane was unsuccessful. Numerous side reactions interfere with the reaction, preventing product formation. Subsequent purification and isolation of the product is likewise made more difficult by the positive charge. This meant it was not possible to successfully verify the target structure.

### Example 9 Polymeric Anthraquinone Dyes

The synthesis of additional polymeric structures based on the compound N,N′-(((anthraquinone-1,4-diyl)bis(azanediyl))bis(propane-3,1-diyl))bis(N,N-dimethyldecan-1-aminium) bromide (7c) was attempted. When used in the body, polymers prevent diffusion into deeper cell layers, thereby preventing toxic effects. Because of the high reactivity of difluoroanthraquinone toward primary amines, polyvinylamine (PVA) was used as the polymeric base structure. To attach the dye to the polymer, a monofunctionalized derivative was accordingly used that is formed as a side product in the synthesis of compound 5 and can be isolated in clean form through purification by column chromatography. Use of equimolar amounts of the two starting materials as shown in Scheme 6 allows compound 20 to be isolated in good yields.

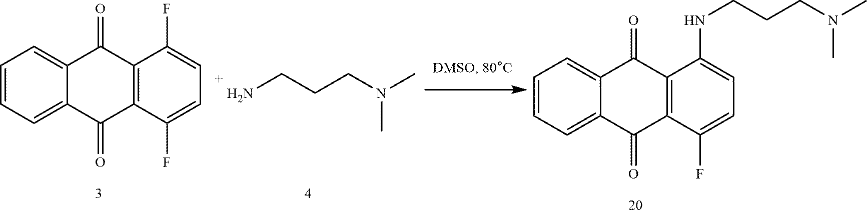

Scheme 6: Synthesis of the monofunctionalized dye 1-((3-(dimethylamino)propyl)amino)-4-fluoroanthraquinone (20)

The successful synthesis of compound 20 was verified by 1H NMR and ¹³C NMR spectroscopy. By comparison with the difunctionalized derivative, the aromatic protons vicinal to fluorine can be shown unambiguously to give rise to two signals, as they are not chemically equivalent. In addition, the signals show higher multiplicity, as coupling to the fluorine atom can likewise be observed. In the ¹H NMR spectrum, the hydrogen atom vicinal to the fluorine atom shows two ³J couplings, whereas the second hydrogen atom gives rise both to a ³J coupling and a ⁴J coupling. This allows unambiguous assignment of the signals.

Further reaction was in analogous manner to compounds 7a-d. This was accomplished by reacting the monofunctionalized dye 20 with 1-bromodecane (6c) in acetone. Here too, the product precipitates in the reaction and can be separated off by filtration. The successful synthesis was verified by ¹H NMR spectroscopy. The reaction is shown in Scheme 7.

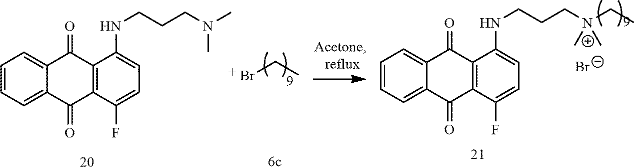

Scheme 7: Synthesis of monofunctionalized dye 21

Attachment to a polymeric structure was achieved by virtue of the free fluorine atom in compound 21 via the primary amines in the polyvinylamine (PVA). The reaction is carried out in THF/H₂O containing traces of triethylamine, to prevent possible protonation of the amino groups. The polymer-analogous attachment to 21 via the amine means that the polymer formed is subsequently blue. The reaction scheme is shown below.

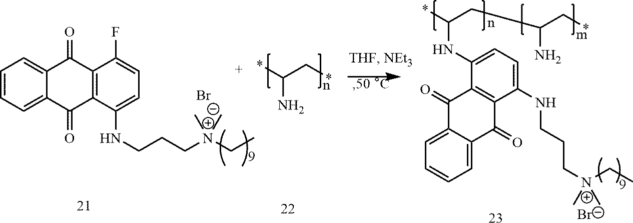

Scheme 8: Reaction of monofunctionalized compound 21 with PVA (22)

The anthraquinone dye was incorporated into the polymer in contents of 10% and 20% by weight. The course of the reaction can be monitored with the naked eye, through the color change brought about by the substitution, and by thin-layer chromatography. The incomplete conversion means that the polymer still contains free amino groups, which interfere both with binding to the tissue to be stained and with GPC monitoring of the molecular weight. No further methods of analysis were therefore employed. The evaluation of binding strength is shown in section 3.1.2.

1. A tissue-targeting complex comprising
a) at least one targeting element [A-L-Q⁺-Alk B⁻] in which A is an anchor group, L a linker, Q⁺ a quaternary ammonium group, B⁻ an ophthalmologically acceptable counterion, and Alk an alkyl chain having a chain length of 4 to 12 carbon atoms;
b) at least one chromophore and/or at least one carrier molecule; for binding to ocular tissue.

2. The tissue-targeting complex as claimed in claim 1, characterized in that the anchor group is a heteroatom or a functional group.

3. The tissue-targeting complex as claimed in claim 1, characterized in that the anchor group is an amino group -(R¹)N- in which R¹ is H or a C₁-C₄ alkyl group and the linker is -C₁-C₆ alkyl.

4. The tissue-targeting complex as claimed in claim 1, characterized in that -Q⁺- is -(R²)(R³)N⁺- in which R² and R³ are each independently C₁-C₄ alkyl radicals.

5. The tissue-targeting complex as claimed in claim 1, characterized in that the counterion B⁻ is a halide ion, hydrogen phosphate ion or hydrogen sulfate ion.

6. The tissue-targeting complex as claimed in claim 1, characterized in that the chromophore is an ophthalmologically tolerated, water-soluble dye.

7. The tissue-targeting complex as claimed in claim 1, characterized in that the chromophore is a compound of the formula I 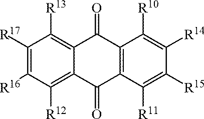
wherein each of the radicals R¹⁰, R¹¹, R¹², and R¹³ is independently hydrogen, C₁-C₄ alkyl radicals, an anchor group as defined in claim 1 or a linker molecule,
each of the radicals R¹⁴, R¹⁵, R¹⁶, and R¹⁷ is independently hydrogen, a C₁-C₄ alkyl radical or a linker unit.

8. The tissue-targeting complex as claimed in claim 1, characterized in that the chromophore is a compound having formula I in which R¹⁰ and R¹¹ are each a targeting element and R¹², R¹³, R¹⁴, R¹⁵, R¹⁶, and R¹⁷ are each hydrogen.

9. The tissue-targeting complex as claimed in claim 6, characterized in that R10 and R11 are each a targeting element, one of radicals R12, R13, R14, R15, R16, and R17 is a linker unit, and the remaining radicals are each hydrogen.

10. The tissue-targeting complex as claimed in claim 1, characterized in that the complex includes at least one carrier molecule, wherein at least one chromophore and at least one targeting element are attached to the carrier molecule.

11. The tissue-targeting complex as claimed in claim 1, characterized in that the targeting element and chromophore are each independently attached to the carrier molecule.

12. The tissue-targeting complex as claim 1, characterized in that at least one targeting element is attached to a chromophore, wherein the carrier molecule includes chromophores and targeting elements attached independently of one another and/or attached chromophores with carrier molecules attached thereto.

13. The tissue-targeting complex as claimed in claim 1, characterized in that the carrier molecule is a water-soluble, ophthalmologically acceptable polymer bearing functional groups for the attachment of chromophore units and/or targeting units.

14. The tissue-targeting complex as claimed in claim 1, characterized in that the carrier molecule is a linear, branched, or bead-type, water-compatible polymer.

15. The tissue-targeting complex as claimed in claim 1, characterized in that the carrier molecule is a homopolymer or copolymer derived from vinylamine.

16. The tissue-targeting complex as claimed in claim 1 for binding to ocular membranes.

17. The tissue-targeting complex as claimed in claim 1 for staining ocular tissue, in particular ocular membranes.

18. Use of the tissue-targeting complex as claimed in claim 1 for binding to ocular membranes and/or for staining ocular membranes, such as epiretinal membranes (ERM) or the inner limiting membrane (ILM).
